(19) United States
(12) Patent Application Publication
(10) Pub. No.: US 2026/0137553 A1
(43) Pub. Date: May 21, 2026

(54) Facial Cold Plunge Device with Integrated Chilling, Filtration, and Breathing Apparatus

(71) Applicant: Nick Reed, Sheridan, WY (US)

(72) Inventor: Nick Reed, Sheridan, WY (US)

(21) Appl. No.: 18/955,946

(22) Filed: Nov. 21, 2024

(51) Int. Cl. A61F 7/00

(52) U.S. Cl. CPC A61F 7/0085; A61F 2007/0003

(57) ABSTRACT

A facial cold plunge device designed to enable prolonged and effective facial immersion in cold water for therapeutic, cosmetic, and wellness purposes. The device comprises an insulated housing unit with an ergonomically contoured reservoir, a chilling mechanism to maintain water temperatures below 50° F., and a thermostat-controlled interface for precise temperature adjustment. A closed-loop water circulation system, incorporating a filtration assembly, ensures consistent cooling and hygienic water conditions. The invention features a retractable breathing apparatus with a snorkel-like tube and a two-way check valve, allowing users to sustain facial submersion without interrupting respiration. The compact and portable design, combined with integrated chilling, filtration, and respiratory support systems, provides a convenient, efficient, and accessible solution for cold therapy, addressing the limitations of traditional full-body cold plunges and DIY facial immersion methods. Applications include stress reduction, vagus nerve stimulation, inflammation control, and various cosmetic benefits.

### BACKGROUND OF THE INVENTION

### FIELD OF INVENTION

The present invention relates generally to devices and systems for cold therapy, particularly to portable, compact apparatuses designed for facial immersion in cold water. More specifically, this invention pertains to a facial cold plunge device with integrated chilling, filtration, and a retractable breathing apparatus, enabling prolonged and convenient cold water immersion for therapeutic, cosmetic, and wellness applications. It finds utility in the health, wellness, and beauty industries, providing users with improved access to the physiological and aesthetic benefits of targeted cold therapy.

### BRIEF SUMMARY OF THE INVENTION

The present invention provides a novel apparatus for facilitating cold water immersion therapy specifically targeting the face. The device comprises an ergonomic bowl-shaped reservoir for water immersion, an integrated chilling mechanism to maintain a user-defined temperature below 50° F., and a filtration system to ensure the water remains clean and hygienic for repeated use. A retractable breathing apparatus is operably affixed to the device, allowing users to sustain facial submersion while maintaining respiration, thereby enabling extended immersion durations without discomfort or interruption.

The invention is compact and portable, making it suitable for residential and commercial use, including settings with spatial or accessibility constraints. The device further incorporates an adjustable thermostat for precise temperature control and a continuous water circulation system for consistent cooling. This invention addresses shortcomings of existing cold therapy methods by eliminating the need for external ice, frequent water changes, and manual temperature adjustments, while providing a safer, more convenient, and effective method for engaging in cold therapy.

The invention is particularly applicable in health and wellness industries, providing benefits such as stress reduction, vagus nerve stimulation, inflammation reduction, and improved skin tone and texture. The invention also offers a cost-effective and space-saving alternative to full-body cold plunges and DIY facial immersion solutions.

### BRIEF DESCRIPTION OF THE FIGURES

FIG. 1: illustrates the key components of the facial cold plunge device, including the adjustable thermostat (102), filter (106), UV light (108), removable mouthpiece for cleaning (110), lowering attached breathing mechanism (112), and GFCI outlet (114), highlighting its functional and safety features.

FIG. 2: The operational flow of the facial cold plunge device involves preparing the reservoir, activating and configuring the device, engaging its chilling and filtration systems, positioning the breathing apparatus, safely immersing the face, and performing post-use maintenance to ensure continued functionality.

### DETAILED DESCRIPTION

The present invention is directed to a novel and advanced facial cold plunge device, which integrates a suite of technical features to enable prolonged and effective facial immersion in cold water for therapeutic, cosmetic, and wellness applications. The invention comprises a main housing unit fabricated from thermally insulated materials, such as high-grade thermoplastics or stainless steel, designed to maintain the thermal integrity of the internal water reservoir.

The housing contains an ergonomically contoured, bowl-shaped reservoir specifically designed to conform to the user's facial profile, enhancing comfort and immersion efficiency while minimizing water spillage. Embedded beneath the reservoir is an integrated chilling mechanism employing a miniature compressor or thermoelectric cooling system.

This system incorporates evaporative cooling coils positioned strategically to ensure uniform cooling across the reservoir, maintaining water temperatures below 50° F.

The chilling mechanism is controlled via an electronic thermostat interface, equipped with a digital display for real-time temperature readings and programmable settings, allowing the user to customize and maintain precise water temperatures for optimal therapeutic effect.

The device further incorporates a closed-loop water circulation system powered by a compact, low-energy pump housed within the base unit. This pump continuously circulates water through a filtration assembly, which includes replaceable carbon or HEPA filters to remove impurities, bacteria, and debris, ensuring the water remains clean and suitable for repeated use without requiring frequent draining or replacement.

The circulation system also incorporates insulated conduits to prevent thermal losses during operation, ensuring consistent and efficient cooling. A unique feature of the invention is the retractable breathing apparatus, which allows users to sustain facial submersion for extended durations without the need to interrupt the immersion process to breathe. This apparatus comprises a snorkel-like breathing tube constructed of medical-grade silicone, which is mounted on the housing unit adjacent to the reservoir.

The tube is mechanically retractable via a spring-based or tensioned arm mechanism, allowing it to be stowed when not in use. The breathing tube is further equipped with a two-way check valve system that regulates airflow, preventing water ingress while facilitating effortless inhalation and exhalation during submersion. The height and position of the breathing tube can be adjusted to align with the user's facial orientation, enhancing comfort and usability.

The invention's structural and functional design prioritizes portability and convenience. The housing unit is compact and lightweight, enabling the device to be easily placed in residential or commercial settings, including small apartments or high-rise buildings where space and access to full-body cold plunges may be limited. Rubberized feet or suction cups on the base provide stability during use, while a detachable power cord or optional battery-operated configuration enhances placement flexibility.

Operationally, the device is user-friendly and efficient. Users fill the reservoir with water to a designated level, activate the chilling and filtration systems, and set their desired water temperature via the thermostat. Once the water reaches the target temperature, the user submerges their face into the reservoir, with the ergonomic design ensuring a secure and comfortable fit. For extended immersions, the user employs the breathing apparatus, allowing for continuous respiration while benefiting from the therapeutic effects of cold water immersion.

The present invention provides significant advantages over existing solutions, addressing numerous limitations in traditional facial cold therapy methods. The integrated chilling system eliminates the need for external ice and the associated waiting periods for cooling, while the filtration system ensures hygienic water conditions without requiring daily maintenance or water changes. The retractable breathing mechanism offers a hands-free solution to the common challenge of holding one's breath during immersion, enabling longer, uninterrupted sessions.

Yet another aspect of the invention is the compact design, which allows for placement in diverse environments, making the invention accessible to individuals who may not have the space or resources for full-body cold plunges. Therapeutic benefits include stimulation of the vagus nerve via the mammalian diving reflex, reduction of stress and anxiety, decreased inflammation, and cosmetic improvements such as reduced puffiness, enhanced skin tone, and minimized signs of aging. The precise temperature control ensures consistent results, a significant improvement over the variability of DIY methods, where the temperature often fluctuates due to melting ice or inconsistent water chilling.

This invention introduces a groundbreaking approach to facial cold therapy, combining state-of-the-art technology with ergonomic and aesthetic design. The integration of chilling, filtration, and respiratory support systems into a single, portable unit offers unparalleled convenience and effectiveness. By providing clean, cold water at a consistent temperature with a seamless user experience, this device sets a new standard for cold therapy applications in the health, wellness, and beauty industries. Its innovative design and advanced technical features make it uniquely positioned to address the unmet needs of individuals seeking accessible, efficient, and effective cold therapy solutions.

### DETAILED DESCRIPTION OF FIGURES

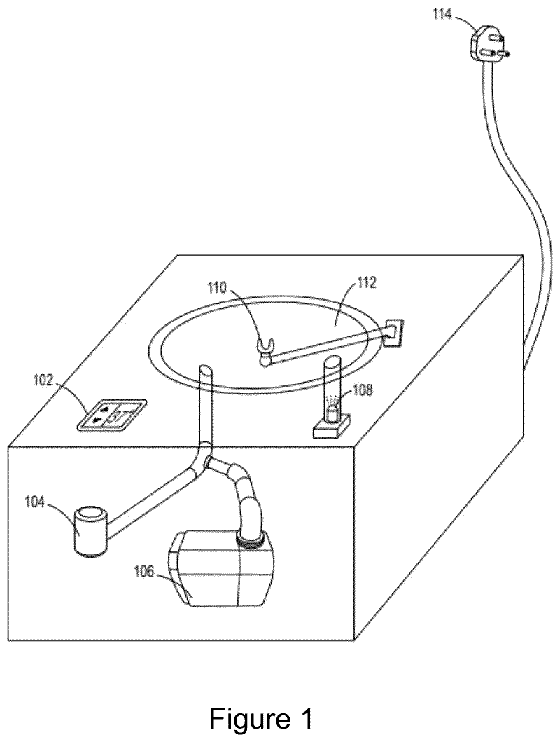

FIG. 1: illustrates the structural and functional components of the facial cold plunge device, showcasing its innovative features that facilitate effective and user-friendly operation. The following components are labeled with their respective reference numerals:

### 102: Adjustable Thermostat

The adjustable thermostat, integrated into the device, allows users to precisely set and maintain the desired water temperature. The thermostat includes a digital interface displaying real-time temperature readings and programmable controls for custom settings.

### 106: Filter

The filtration system, identified as component 106, ensures water hygiene by removing impurities, bacteria, and debris. The filter is replaceable and part of the closed-loop water circulation system, maintaining the cleanliness of the water for multiple uses.

### 108: UV Light

Positioned adjacent to the filtration system, the UV light sterilizes the circulating water by eliminating microbial contaminants, further enhancing water purity and safety during prolonged immersion.

### 110: Removable Mouthpiece for Easy Cleaning

The mouthpiece of the breathing apparatus, designated as 110, is detachable to facilitate thorough cleaning. Constructed from medical-grade silicone, it ensures hygiene and comfort during repeated usage.

### 112: Lowering Attached Breathing Mechanism

The attached breathing mechanism, referenced as 112, is a retractable snorkel-like tube that lowers into position for use. This component includes an adjustable height mechanism and a two-way check valve to allow seamless respiration while preventing water ingress during facial immersion.

### 114: GFCI Outlet

The Ground Fault Circuit Interrupter (GFCI) outlet, labeled 114, ensures electrical safety during operation, protecting the user and device from potential electrical faults, particularly in wet environments.

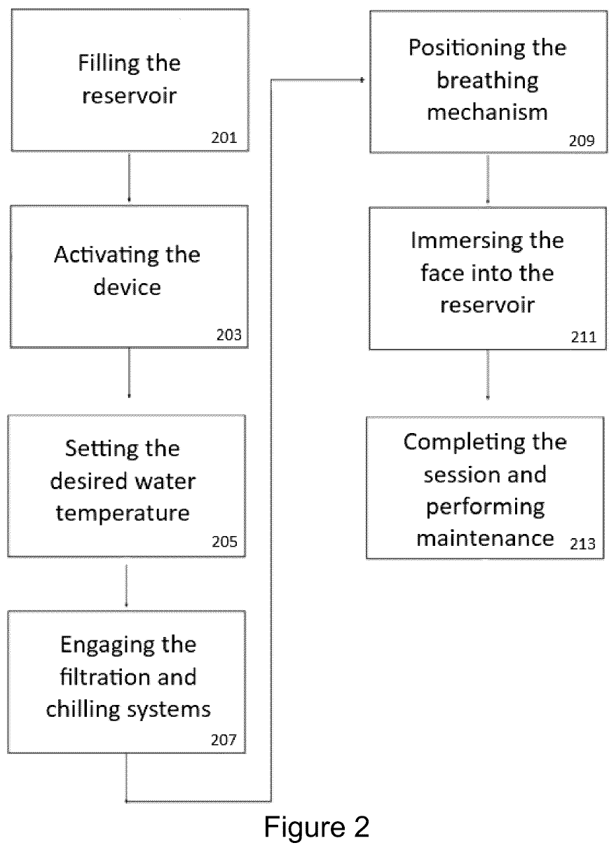

### FIG. 2.201: Filling the Reservoir

The first step involves filling the ergonomically contoured reservoir with water to the indicated fill line. The reservoir is designed to accommodate an optimal volume for immersion, ensuring sufficient water depth for effective facial coverage while minimizing overflows or spillage.

FIG. 2.203: Activating the Device-After the reservoir is filled, the device is powered on by connecting it to a power source and engaging the GFCI outlet for safety. The internal systems, including the thermostat, filtration, and chilling mechanisms, initialize upon activation, preparing the device for operation.

FIG. 2.205: Setting the Desired Water Temperature-Using the adjustable thermostat, the user selects the desired water temperature. The thermostat interface displays real-time temperature readings and allows for precise adjustment to achieve optimal cooling below 50° F. This step ensures consistent temperature settings for effective cold therapy.

FIG. 2.207: Engaging the Filtration and Chilling Systems-Once the temperature is set, the water circulation begins as the pump activates, channeling water through the filtration assembly to remove impurities. Simultaneously, the chilling mechanism, utilizing a compressor or thermoelectric system, ensures the water cools uniformly to the pre-selected temperature.

FIG. 2.209: Positioning the Breathing Mechanism-The retractable breathing apparatus is adjusted for the user's facial alignment. The snorkel-like tube lowers into position via its spring-loaded mechanism, and the user aligns the mouthpiece for comfortable respiration. The two-way check valve prevents water ingress while facilitating seamless airflow.

FIG. 2.211: Immersing the Face into the Reservoir-The user submerges their face into the cold water reservoir, ensuring a snug fit against the ergonomic contours of the reservoir to minimize spillage. The breathing apparatus enables prolonged immersion without the need to interrupt for breaths, optimizing the therapeutic effects of the session.

FIG. 2.213: Completing the Session and Performing Maintenance-Upon completion, the user removes their face from the reservoir, powers off the device, and detaches the mouthpiece for cleaning. The filtration assembly is inspected, and any necessary water replacements are made, ensuring the device remains clean and operational for future use.

What is claimed is:

1) A facial cold plunge device, comprising:
a) a housing unit fabricated from thermally insulated material;
b) an ergonomically contoured, bowl-shaped reservoir integrated into the housing unit and configured to accommodate a user's face for water immersion;
c) a chilling mechanism operatively connected to the reservoir, the chilling mechanism comprising evaporative cooling coils and a compressor or thermoelectric cooling system for maintaining water temperature at a user-defined level below 50°F;
d) a thermostat control system operably connected to the chilling mechanism, the thermostat system comprising a user interface with a digital display and
programmable temperature settings for precise control of water temperature;
e) a water circulation system, comprising:
i) a pump configured to circulate water within the reservoir;
ii) insulated conduits connecting the pump to the reservoir; and
iii) a filtration assembly including a replaceable filter configured to remove impurities and maintain hygienic water conditions.
f) a retractable breathing apparatus mounted to the housing unit, the breathing apparatus comprising:
i) a snorkel-like breathing tube constructed from medical-grade silicone;
ii) a two-way check valve for regulating airflow and preventing water ingress; and
iii) an adjustable positioning mechanism for aligning the breathing tube with a user's facial orientation during water immersion; and
g) a power source operatively connected to the chilling mechanism, pump, and thermostat control system, the power source configured to enable continuous operation.

2) The facial cold plunge device of claim 1, wherein the reservoir is contoured to provide a watertight seal against the user's face to minimize water spillage during use.

3) The facial cold plunge device of claim 1, wherein the chilling mechanism is operable to cool water within the reservoir to temperatures as low as 32°F while maintaining uniform cooling across the reservoir surface.

4) The facial cold plunge device of claim 1, wherein the filtration assembly comprises a carbon or HEPA filter capable of removing bacteria, debris, and other impurities from the water during operation.

5) The facial cold plunge device of claim 1, wherein the retractable breathing apparatus is operatively mounted on a spring-loaded mechanism to allow stowing when not in use and deployment during immersion.

6) The facial cold plunge device of claim 1, wherein the two-way check valve of the breathing apparatus is configured to allow seamless inhalation and exhalation while submerged in water.

7) The facial cold plunge device of claim 1, wherein the thermostat control system includes a programmable timer function to automatically maintain the desired water temperature over a predefined period.

8) The facial cold plunge device of claim 1, wherein the housing unit comprises a compact, lightweight design with rubberized feet or suction cups for enhanced stability during operation.

9) The facial cold plunge device of claim 1, further comprising an optional battery-powered mode to enable portable and off-grid use.

10) The facial cold plunge device of claim 1, wherein the water circulation system is configured to operate continuously to maintain consistent water temperature and filtration during extended periods of use.

11) The facial cold plunge device of claim 1, wherein the breathing apparatus is adjustable in length and height to accommodate users of varying facial dimensions and preferences.

12) The facial cold plunge device of claim 1, wherein the ergonomic reservoir is constructed with non-slip interior coatings to enhance user safety and comfort during immersion.

13) The facial cold plunge device of claim 1, wherein the digital display of the thermostat system provides real-time feedback on water temperature and system status.

14) The facial cold plunge device of claim 1, further comprising an integrated safety mechanism configured to shut down the chilling mechanism and pump in the event of water leakage or overheating.

15) The facial cold plunge device of claim 1, wherein the device is configured to support detachable accessories, including alternative filtration systems and enhanced breathing apparatus configurations.

16) These claims define the invention comprehensively while highlighting the novel features and operational advantages of the facial cold plunge device. Let me know if adjustments or additional claims are required.
